(19) United States
(12) Patent Application Publication
(10) Pub. No.: US 2015/0025354 A1
(43) Pub. Date: Jan. 22, 2015

(54) DEVICE FOR MEASURING ELECTROPHYSIOLOGICAL SIGNALS AND A MANUFACTURING METHOD FOR MANUFACTURING THE DEVICE

(71) Applicants: Kenneth Alexander Johannes Salonius, Helsinki (FI); Riku Oskari Lehtomäki, Helsinki (FI)

(72) Inventors: Kenneth Alexander Johannes Salonius, Helsinki (FI); Riku Oskari Lehtomäki, Helsinki (FI)

(21) Appl. No.: 14/333,043

(22) Filed: Jul. 16, 2014

(30) Foreign Application Priority Data
Jul. 18, 2013 (FI) 20135781

(51) Int. Cl. A61B 5/00; A61B 5/0478; A61B 5/0492; A61B 5/0408

(52) U.S. Cl. CPC A61B 5/6805; A61B 5/04085; A61B 5/0478; A61B 5/0492
USPC 600/389; 600/393; 600/388; 29/846

(57) ABSTRACT

A device (100) for measuring electrophysiological signals of a body comprising electrodes (101) and a multilayer supporting medium (102), such as garment, like a belt, for supporting said electrodes (101). The multilayer supporting medium (102) comprises at least one stretchable layer (103) and one non-stretchable (104) corrugated (104a) layer. The layers are coupled with each other in numerous portions (105) so that the corrugation portions (104a) of said non-stretchable corrugated layer (104) between the coupling portions (105) are free from said stretchable layer (103). The electrodes (101) are also arranged into the non-stretchable layers (104) at the coupling portions (105).

### TECHNICAL FIELD OF THE INVENTION

The invention relates to a device for measuring electrophysiological signals and a manufacturing method for manufacturing the device. Especially the invention related to structural improvements of the device as an electrode carrier and cable assemblies for improved usability and manufacturability.

### BACKGROUND OF THE INVENTION

Widely used electrophysiological measurements include for example electroencephalography, electrocardiography, and electromyography. All electrophysiological measurements include placement of plurality of electrodes on a skin, ranging typically from 2 to over 256 to measure. The need for increasing the amount of electrode increases with the need of more accurate measurements, such as knowing spatial differences of voltages measured on the skin in connection with electrical impedance tomography (EIT) measurements as an example.

All electrophysiological operations need electrodes placed on the skin and these electrodes need conductive paths connecting the electrodes to the measurement device. For example electrical impedance tomography devices use great amount of electrodes, typically 16 and ranging even to over 256. Due to the requirement of great amount of electrodes, the amount of wiring needed to connect the electrodes in a measuring device, such as a belt in the EIT device is also extensive.

In addition, traditionally in electrophysiological measurements, adhesive electrodes are used by placing them on the skin one-by-one and connected to the device with separate cables one-by-one. The requirement of cables placing and installation in correct positions has been addressed by having trained nurses to place the electrodes and the cables. However, due to operation of placing the electrodes and connecting the cables has limited the application of electrophysiological measurements to bedside monitoring of patients or patients otherwise in immobile positions.

There are however some disadvantages relating to the known prior art, such as the placement of the plurality of electrodes, as well as the extensive number of cables limiting the movement of the person connected to the measurement device. Furthermore the cables between the electrode and the device might be subject to stress loosening the connection between the measuring device and the electrode thus causing unnecessary failure risks of the measuring device.

In addition there are also some disadvantages relating to the cable solutions in belt like structures, such as structural weakness related to stretching the belt near the maximum length. When the belt or the like is stretched to the maximum length, the applied force causes stress to the wires easily damaging the conductive paths and causing failure of the electrode. Moreover due to the great amount of electrodes on the measuring device, such as the belt, the amount of wiring needed to connect these is also extensive. This sets limitations to manufacturability of such assemblies using the traditional methods such as integrated single wires or multi wire cable bundles.

### SUMMARY OF THE INVENTION

An object of the invention is to alleviate and eliminate the problems relating to the known prior art. Especially the object of the invention is to provide a device and manufacturing method of the device so that correct placement of a plurality of electrodes of the device on the skin or object to be measured is easy and fast. In addition an object is to avoid the extensive number of cables limiting the movement of the person connected to the measurement device. Moreover an object is to minimize or even remove any harmful forces causing stress to the structure of the device or especially to the electrically conducting wires or other conductive paths and thereby causing failure of the electrodes of the device.

The object of the invention can be achieved by the features of independent claims.

The invention relates to a device for measuring electrophysiological signals of a body according to claim 1. In addition the invention relates to a manufacturing method of the device according to claim 6.

According to an embodiment of the invention a device for measuring electrophysiological signals, such as e.g. pulse or other electroencephalography, electrocardiography, or electromyography related signals of a body, comprises electrodes for measuring said signals from the body. In addition the device advantageously comprises a multilayer supporting medium, such as a garment, for supporting said electrodes. The multilayer supporting medium comprises at least one stretchable layer and at least one non-stretchable corrugated layer, said layers being coupled with each other in numerous portions so that the corrugation portions of said non-stretchable corrugated layer are provided between the coupling portions. In addition the corrugation portions of the non-stretchable corrugated layer are configured to be free from the stretchable layer.

Furthermore the electrodes are arranged into the non-stretchable layers at the coupling portions, whereupon any possible external forces and stresses against the electrodes are minimized.

In addition, according to an embodiment, the non-stretchable layer advantageously comprises conductive paths for transferring measured electric signal from the electrodes. When the conductive paths are provided into or onto the non-stretchable layer as described in this document, the device is still stretchable but any interactions of possible external forces and stresses against the conductive paths are minimized or even eliminated.

It is to be noted that the device may, according to additional embodiments, also comprise a controlling unit for controlling the measurements, as well as a communication means for communicating at least portion of the measurements outside the device, for example using Bluetooth techniques or other known by the skilled person. Again, according to an exemplary embodiment, the device may also comprise other additional electrodes, such as injecting electrodes configured to inject electric current to the body, as is typically the case with the EIT devices. In that exemplary case the measuring electrodes can be configured to measure the resulting voltage as said resulting signal on the surface of said object.

According to an advantageous embodiment the device or its multilayer supporting medium may be implemented by or integrated to or comprises a garment, e.g. belt, harness, shirt, bra, strap, or vest, as an example.

In addition the invention relates also to a manufacturing method for manufacturing the device described in this document. According to an embodiment the manufacturing method comprises steps of:
providing a first stretchable layer,
providing a second non-stretchable layer,
coupling said stretchable and non-stretchable layers with each other at numerous portions so to provide corrugation portions of said non-stretchable corrugated layer between said coupling portions, where said corrugated portions of said non-stretchable layer between said coupling portions are free from said stretchable layer, and
providing electrodes at said coupling portions in connection with said non-stretchable layer and electrically connecting said electrodes with said conductive paths.

Also electrically conductive paths may be provided into said second non-stretchable layer. It is to be noted that it might be advantageous to have the second non-stretchable layer longer than said stretchable layer in rest. In addition, according to an embodiment, the couplings of the stretchable and non-stretchable layers, as well as also attaching of the electrodes to the device, may be implemented by laminating, gluing, sewing and/or riveting, for example. Moreover the electrodes and/or electrically conductive paths advantageously comprise electrically conductive fibres.

The present invention offers advantages over the know prior art, such as improves the usability of connecting measurement electrodes to electrophysiological measurement devices by removing the need for separate cables between electrodes and the measurement device. Furthermore, unlike in the typical prior art, where adhesives and conductive gels are typically required when using the measuring device implemented e.g. by a belt structure, the stretchable nature of the device according to the invention ensures high quality contact between the electrodes and the body. This is another highly preferable feature outside the hospital and ambulatory environment. In addition the device according to embodiments is very easy, fast and inexpensive to manufacture.

### BRIEF DESCRIPTION OF THE DRAWINGS

Next the invention will be described in greater detail with reference to exemplary embodiments in accordance with the accompanying drawings, in which:

FIG. 1 illustrates a principle of an exemplary device and manufacturing method of manufacturing the device for measuring electrophysiological signals according to an advantageous embodiment of the invention.

### DETAILED DESCRIPTION

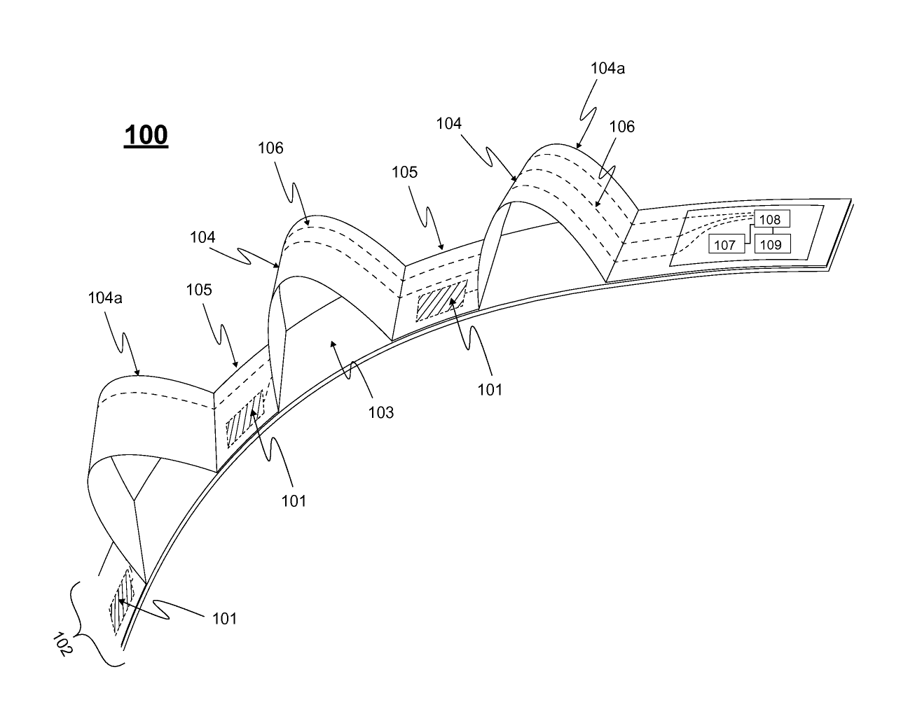

FIG. 1 illustrates a principle of an exemplary device 100 and manufacturing method of manufacturing the device for measuring electrophysiological signals according to an advantageous embodiment of the invention. The device advantageously comprises electrodes 101 for measuring said signals from the body. In addition the device advantageously comprises a multilayer supporting medium 102, such as a garment, and in particularly as a belt, for supporting said electrodes, for example. The multilayer supporting medium 102 comprises at least one stretchable layer 103 and at least one non-stretchable 104 corrugated layer. The layers 103, 104 are advantageously coupled, such as laminated, with each other in numerous portions 105 so that the corrugation portions 104a of said non-stretchable corrugated layer 104 are provided between the coupling portions 105. In addition the corrugation portions of the non-stretchable corrugated layer are configured to be free from the stretchable layer 103. Furthermore the electrodes 101 are arranged into the non-stretchable layer 104 at the coupling portions 105, whereupon any possible external forces and stresses against the electrodes are minimized.

The non-stretchable layer 104 advantageously comprises conductive paths 106 for transferring measured electric signal from the electrodes 101.

It is to be noted that the device may, according to additional embodiments, also comprise a power source 107, controlling unit 108 for controlling the measurements, as well as a communication means 109 for communicating at least portion of the measurements outside the device, for example using Bluetooth techniques or other known by the skilled person.

The device is advantageously manufactured by
providing a first stretchable layer 103,
providing a second non-stretchable layer 104, and
coupling said stretchable 103 and non-stretchable 104 layers with each other at numerous portions 105 so to provide corrugation portions 104a of said non-stretchable corrugated layer 104 between said coupling portions 105, and so that said corrugated portions 104a of said non-stretchable layer 104 between said coupling portions 105 are free from said stretchable layer 103

In addition electrodes are advantageously provided, e.g. laminated or otherwise attached, at said coupling portions 105 in connection with said non-stretchable layer 104. Furthermore also conductive paths 106 are provided into or onto the non-stretchable layer 104, whereafter also the electrodes 101 are electrically connected with the conductive paths 106.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

1. A device for measuring electrophysiological signals of a body, the device comprising:
electrodes for measuring the signals from the body, and
a multilayer supporting medium for supporting said electrodes, where said multilayer supporting medium comprises at least one stretchable layer and one non-stretchable corrugated layer, said layers being coupled with each other in numerous portions so that the corrugation portions of said non-stretchable corrugated layer between the coupling portions are free from said stretchable layer and wherein said electrodes are arranged into the non-stretchable layer at the coupling portions.

2. A device of claim 1, wherein the non-stretchable layer comprises conductive paths for transferring measured electric signal from the electrodes.

3. A device of claim 1, wherein the device comprises a controlling unit for controlling the measurement and a communication means for communicating at least portion of the measurements.

4. A device of claim 1, wherein the device comprises also injecting electrodes configured to inject electric current to the body, and where said measuring electrodes are configured to measure resulting voltage as a resulting signal on the surface of said body.

5. A device of claim 1, wherein said device or multilayer supporting medium comprises a garment, e.g. belt, harness, shirt, bra, strap, or vest.

6. A manufacturing method for manufacturing a device according to any of previous device claims, wherein the method comprises steps of:
providing a first stretchable layer,
providing a second non-stretchable layer,
coupling said stretchable and non-stretchable layers with each other at numerous portions so to provide corrugation portions of said non-stretchable corrugated layer between said coupling portions, where said corrugated portions of said non-stretchable layer between said coupling portions are free from said stretchable layer, and
providing electrodes at said coupling portions in connection with said non-stretchable layer and electrically connecting said electrodes with said conductive paths.

7. A manufacturing method of claim 6, wherein the method further comprises providing electrically conductive paths into said second non-stretchable layer,

8. A manufacturing method of claim 6, wherein said second non-stretchable layer is longer than said stretchable layer in rest.

9. A manufacturing method of claim 6, wherein said couplings of said stretchable and non-stretchable layers are implemented by laminating, gluing, sewing and/or riveting.

10. A manufacturing method of claim 6, wherein said electrodes and/or electrically conductive paths comprise electrically conductive fibres.
